(19) United States
(12) Patent Application Publication
(10) Pub. No.: US 2015/0283098 A1
(43) Pub. Date: Oct. 8, 2015

(54) PRENYLATED CHALCONE FORMULATION FOR THE TREATMENT OF BEES

(71) Applicant: BIOACTIVE-TECH SP. Z O.O., Radosci (PL)

(72) Inventor: Mariusz Gagos, Radosci (PL)

(21) Appl. No.: 14/436,343

(22) PCT Filed: Aug. 22, 2013
(86) PCT No.: PCT/IB2013/001829

§ 371 Date: Apr. 16, 2015

(30) Foreign Application Priority Data
Oct. 16, 2012 (PL) P.401225

(51) Int. Cl. A61K 31/12; C07C 49/796; A61K 45/06

(52) U.S. Cl. CPC A61K 31/12; A61K 45/06; C07C 49/796

(57) ABSTRACT

The invention provides a formulation preventing the collapse of insects of Apidae family, especially bees and bumblebees, especially as a result of a syndrome specified as CCD (Colony Collapse Disorder) and the use thereof. Moreover, the formulation increases vigour in a bee colony. The invention belongs to the field of apiary and protection of environment from negative effects of using insecticides in agriculture and fruit culture.

### TECHNICAL FIELD

The invention provides a formulation preventing collapse of insects of Apidae family and uses thereof. The invention belongs to the field of beekeeping and protection of the environment from negative effects of the use of insecticides in arable farming and fruit culture. The invention makes it possible to prevent the collapse of insects of Apidae family, especially bees and bumblebees and increases their vigour.

### BACKGROUND OF THE INVENTION

The syndrome of colony collapse disorder (CCD) occurring in the post-productive period is a world-wide problem. It is particularly severe in highly industrialized countries. Statistical data indicates that CCD may cause death even of 90% of bee colonies in an apiary. It results not only in a drastic decrease in honey production but, first of all, high losses in agricultural production.

A breakthrough in understanding CCD was made by the reports of French bee-keepers in 1993 on mass collapse of bee colonies after the use of insecticides from the group of neonicotinoids for sunflower seed pickling. Researchers from the National Institute for Agricultural Research in Avignon found that one of the causes of CCD, apart from Nosema, is the use of imidaclopride in agriculture a compound of neonicotinoids group, (Environmental Microbiology (2010, 12(3). 774-782). This compound is a neurotoxine, i.e. it acts on the nervous system of insects causing, among other things, their disorientation, and finally death. It is widely used as a component of seed dressing to protect cultivable plants from pests, and as an active substance of many formulations that are used directly on plants.

The main target of neonicotinoids action is the nervous system of insects. Neonicotinoids are compounds structurally and functionally similar to nicotine, and due to the chemical structure, they are also referred to as chloronicotinyles. They effectively mimic nicotine action in the central nervous system. In low concentrations, they cause stimulation of cholinergic receptors of the nicotine type and depolarisation of a neuronal membrane, and thus the stimulation state, whereas in higher concentrations, they block synaptic conductance. This state is maintained, because in a synaptic space, there are no esterases decomposing nicotine, or their structural analogues. This leads to a disturbance in neuronal impulse and causes overstimulation of post-synaptic neurones, manifested by seizures and convulsions. Excitotoxicity of nicotine and its analogues leads to impairment of neurones and, consequently, to death of insects.

Apart from the neurologic symptoms, neonicotinoids cause also a decrease in bee and bumblebee resistance to diseases, especially nosemosis caused by parasites of Nosema spp. species.

Up to now, no reports have been found in the literature on an effective method or agent that would allow to eliminate or protect bees from the action of nicotinoide compounds, and especially imidaclopride or thiametoxame used in insecticide formulations and which, at the same time, would allow to eliminate the CCD phenomenon.

The purpose of the studies presented in this application was to select of compounds having the neuroprotective potential which would prevent mass collapse of insects from Apidae family, in particular bee and bumblebee families exposed to insecticides from the neonicotinoide group and other toxins in insects of Apidae family. Another purpose was to improve the vigour and increase of resistance of insects from Apidae family, in particular bees and bumblebees, to diseases occurring as a consequence of pesticides use, and control of nosemosis Nosema spp in insects of Apidae family, mainly Nosema Apis i Nosema Cerenae and other diseases caused by viruses, fungi, bacteria and saprophytes Varroa destructor.

### DISCLOSURE OF THE INVENTION

The subject matter of the invention relates to a formulation preventing collapse of insects of Apidae family and increasing their vigour, which comprises prenylated chalcone derivative or its metabolites of purity about 0.001-100%, a solvent and additional substances admissible in beekeeping, in concentration of prenylated chalcone derivative or its metabolite not less than 0.001 mg per 11 of the formulation.

The subject matter of the invention also relates to a formulation preventing collapse of insects of Apidae family and increasing their vigour characterised in that it comprises a prenylated chalcone derivative or its metabolites of purity about 0.001-100%, a solvent and additional substances admissible in beekeeping, in the concentration of a prenylated chalcone derivative or its metabolite 0.001-1000 mg per ll of the formulation.

As a prenylated chalcone derivative, the formulation contains a compound selected from the group consisting of xanthohumol, xanthohumol B, xanthohumol E, α,β-dihydroxanthohumol, isobavachalcone, xanthohumol C, xanthohumol D, isodehydrocycloxanthohumol, desmethylxanthohumol, xanthogalenol, 4′-O-methylxanthohumol, 3′-geranyl chalconarygenine, 3′,5′-diprenylchalconearygenine, 5′-prenylxanthohumol, flavokavine and/or prenylated derivatives of chalcone.

In a more preferable aspect of the invention, the formulation contains xanthohumol as a prenylated derivative of chalcone.

In a preferable aspect of the invention, the concentration of prenylated derivative of chalcone or its metabolite, preferably xanthohumol, in the formulation is not lower than 0.001 mg/l.

In another aspect of the invention, the concentration of prenylated chalcone derivative or its metabolite in the formulation, preferably xanthohumol, is in the range of about 0.08 mg/l to about 8 g/l.

In another aspect of the invention, the concentration of xanthohumol in the formulation is selected from the group consisting of 0.08 mg/l, 0.1 mg/l, 0.5 mg/l, 1 mg/l, 1.6 mg/l, 2 mg/l, 5 mg/l, 10 mg/l 16 mg/l, 20 mg/l, 30 mg/l, 40 mg/l 50 mg/l, 70 mg/l, 100 mg/l, 200 mg/l, 300 mg/l, 500 mg/l, 1 g/l, 5 g/l lub 8 g/l.

In another aspect of the invention, the concentration of prenylated chalcone derivative or its metabolite, preferably xanthohumol, in the formulation is within the range of 0.08 mg/l to about 1 g/l, more preferably 1-40 mg/l.

The formulation according to the invention comprises xanthohumol of purity from 0.001 to 100%. More preferably, the formulation according to the invention comprises xanthohumol of purity 0.1%, 1%, 5%, 10%, 20%, 26%, 30%, 40%, 50%, 60%, 70%, 80% or preferably at least 90%.

The formulation according to the invention, as a metabolite of prenylated chalcone derivative, comprises a compound selected from the group consisting of isoxanthohumol, desmethylxanthohumol, 6-prenylnaryngenine, 8-prenylnaryngenine, 6,8-prenylnaryngenine, glucuronide derivatives of xanthohumol, products of hydroxylation, O-methylation, 0-acetylation, epoxidation and cyclization of prenyl chain and glucuronidation in position C-4″.

The formulation according to the invention as an additional substance comprises sugar syrup.

Sugar syrups used to prepare a formulation according to the invention may be the solutions used in beekeeping and known in the prior art consisting of sucrose, glucose and fructose commercially available on the market, as well as isoglucose solution which is also available on the market (Apiinwert, Germany; Bioinwert, Pulawy, Poland).

The formulation according to the invention comprises, as an additional substance, a substance selected from the group consisting of other flavonoids and chalcones or their metabolites, coumarins, carotenoids, polyphenols, oxalic acid, formic acid, ascorbic acid, fatty acids, lipids, liposomal forms of the mentioned compounds, proteins, peptides, vitamins, minerals, propolis, honeys, flower pollens, royal jelly, plant oils, derivatives of castor oil, decosahexaene acid, anise or eucalyptus oil, tee extracts, hop extracts, coffee extracts, caffeine or other herbal and vegetable extracts.

In the formulation according to the invention, water solution is used as a solvent, pure alcohol or water ethyl alcohol solution may also be solvents.

In the formulation according to the invention, water solution of DMSO, Kolliphor or Cremophor are also used as solvents.

Kolliphor (previously known as Cremphor) is a family of non-ionic solvents increasing solubility and emulgation. Kolliphor RH 40 is obtained by way of reaction of one mole of hydrogenated castor oil and 45 moles of ethylene oxide, and Kolliphor EL and ELP are formed as a result of reaction of castor oil and ethylene oxide in a molar ratio of 01:35.

The formulation according to the invention, as a solvent, comprises cyclodextrins and/or sugars or complexes with cyclodextrins or sugars. In the formulation, the solvents may be also non-polar solvents. Aqueous solutions being the solvents in the formulation may be preferably alkaline or acid solutions.

In the formulation according to the invention, aqueous solution may preferably be a solution comprising complexes of prenylated chalcone derivative or its metabolite, preferably xanthohumol, with sugars, alkaline and/or transitory earth metals.

The formulation according to the invention may be in the solid or liquid form, for example in the form of sugar syrup, dough for stimulating feeding of insects or in the form of a spray. The formulation according to the invention, if it is in a liquid form, may also be put in the spaces between frames in the hive.

In the solid form the formulation may be in the form of a pure pulverized xanthohumol ready to be solubilized with a solvent, xanthohumol complexes with sugars, lipids, proteins or metals freeze-dried or dried by other methods and ready to be solubilized with a solvent.

The formulation according to the invention may also be delivered to apiaries in the form of a sugar strap or other strap with nourishing substances for biting by insects. Such strap may also be a matrix made of paper or plastics. The formulation according to the invention may be used in the form of straps on the surface on which xanthohumol was adsorbed, on which honey bees enter the hive.

The formulation according to the invention is for administration at a dose of about 0.01 mg to about 1 g per one bee colony.

The invention also relates to the use of a prenylated chalcone derivative or its metabolites in the prevention and/or control of collapse of insects from Apidae family as a result of colony collapse disease (CCD) of bees and bumblebees.

In the use according to the invention, as a prenylated chalcone derivative, a compound selected from the group consisting of xanthohumol, xanthohumol B, xanthohumol E, α,β-dihydroxanthohumol, isobavachalcone, xanthohumol C, xanthohumol D, isodehydroxanthohumol hydrate, desmethylxanthohumol, xanthogalenol, 4′-O-methylxanthohumol, 3′-geranyl chalconenaryngenine, 3′,5′-diprenylchalconearygenine, 5′-prenylxanthohumol, flavokavine and/or other prenylated chalcone derivatives is used.

In the use according to the invention, xanthohumol is used as a prenylated chalcone derivative.

Preferably, as a metabolite of prenylated chalcone derivative, a compound selected from the group consisting of isoxanthohumol, desmethylxanthohumol, 6-prenylonaryngenin, 8-prenylonarygenine, 6,8-prenylonarygenine, glucuronide derivatives of xanthohumol and its metabolites, products of hydroxylation, methylation, acetylation, epoxidation and cyclization of prenyl chain of xanthohumol and its metabolites, sulphone xanthohumol derivatives, products of cyclization of chalcone to flavanone, cyclization of chalcone to aurone and other products obtained as a result of chemical and microbiological attachment of sugars to xanthohumol and its metabolites is used.

In the use according to the invention, a dose of prenylated chalcone derivative or its metabolite, preferably xanthohumol, per one bee colony, is from about 0.01 mg to about 1 g.

According to the use, a chalcone derivative or its metabolite is used in bees and bumblebees. Bees are selected from Apis mellifera and Apis cerenae species.

Preferably, in the use, a formulation is used as mentioned above and defined in the claims.

The subject matter of the invention also relates to the use of a prenylated chalcone derivative or its metabolites for improving the vigour of insects from Apidae family.

Preferably, as a prenylated chalcone derivative, a compound selected from the group consisting of xanthohumol, xanthohumol B, xanthohumol E, α,β-dihydroxyxanthohumol, isobavachalcone, xanthohumol C, xanthohumol D, isodehydrocycloxanthohumol hydrate, desmethylxanthohumol, xanthogalenol, 4′-O-methylxanthohumol, 3′-geranyl chalconaryngenine, 3′,5′-diprenylchalconearyngenine, 5′-prenylxanthohumol, flavokavine and/or other prenylated chalcone derivatives are used, more preferably, as a prenylated chalcone derivative, xanthohumol is used.

In the use according to the invention, as a metabolite of prenylated chalcone derivative, a compound selected from the group consisting of isoxanthohumol, desmethylxanthohumol, 6-prenylonarygenine, 8-prenylonaryngenin, 6,8-prenylonaryngenin, glucuronide derivatives of xanthohumol and its metabolites, products of hydroxylation, methylation, acetylation, epoxidation and cyclization of prenylated chain of xanthohumol and its metabolites, sulphone xanthohumol derivatives, cyclization of chalcone to flavanone, cyclization of chalcone to aurone and other products obtained as a result of chemical and microbiological attachment of sugars to xanthohumol and its metabolites is used.

In the use according to the invention, xanthohumol dose of about 0.01 to about 1 g per one colony from about 0.01 mg to about 1 g is used.

Prenylated chalcone derivative or its metabolite is used in bees and bumblebees. Bees are selected from Apis millifera and Apis cerenae species.

In a preferable aspect of the invention a formulation is used as mentioned above and defined in claims.

The subject matter of the invention also relates to the use of prenylated chalcone derivative or its metabolites for the control of diseases selected from the group of Nosema spp, sacbrood disease, mycosis, Varroosis or other pathogens of insects from Apidae family.

In the use according to the invention, as a prenylated chalcone derivative, a compound selected from the group consisting of xanthohumol, xanthohumol B, xanthohumol E, α,β-dihydroxanthohumol hydrate, isobavachalcone, xanthohumol C, xanthohumol D, isodehydrocycloxanthohumol, desmethylxanthohumol, xanthogalenol, 4′-O-methylxanthohumol, 3′-geranyl chalconearyngenine, 3′5′-diprenylchalconenaryngenine, 5′-prenylxanthohumol, flavocavine and/or other prenylated chalcone derivatives is used.

Preferably, xanthohumol is used as a prenylated chalcone derivative.

As a metabolite of prenylated chalcone derivative, a compound selected from the group consisting of isoxanthohumol, desmethylxanthohumol, 6-prenylonaryngenin, 8-prenylonaryngenin, 6,8-prenylonaryngenin, glucuronide derivatives of xanthohumol and its metabolites, products of hydroxylation, methylation, acetylation, epoxidation and cyclization of prenyl xanthohumol chain and its metabolites, sulphone xanthohumol derivatives, cyclization of chalcone to flavone, cyclization of chalcone to aurone and other products obtained as a result of chemical and microbiological attachment of sugars to xanthohumol and its metabolites is used.

In the use according to the invention, a dose of prenylated chalcone derivative or its metabolite, preferably xanthohumol, per one bee colony is from about 0.01 mg to about 1 g.

Prenylated chalcone derivative in this use is applied in bees, while preferably bees are selected from Apis mellifera and Apis ceranae species.

In the use of the invention, a formulation as mentioned above and defined in claims is used.

The subject matter of the invention also relates to the use of prenylated chalcone derivative or its metabolites for preventing and/or controlling of the effects of the use insecticides from the neonicotinoids group and other toxins in insects of Apidae family.

As a prenylated chalcone derivative in this use, preferably a compound selected from a group consisting of xanthohumol, xanthohumol B, xanthohumol E, α,β-dihydroxyxanthohumol, isobavachalcone, xanthohumol C, xanthohumol D, isodehydrocycloxanthohumol hydrate, desmethylxanthohumol, xanthogalenol, 4′-O-methylxanthohumol, 3′-geranyl chalconaryngenine, 3′,5′-diprenylchalconenaryngenine, 5′-prenylxanthohumol, flavokavine and/or other prenylated chalcone derivatives is used.

Even more preferably, xanthohumol is used as a prenylated chalcone derivative.

Preferably, as a metabolite of prenylated chalcone derivative in the use, a compound selected from a group consisting of isoxanthohumol, desmethylxanthohumol, 6-prenylonaryngenin, 8-prenylonaryngenin, 6,8-prenylonaryngenin, glucuronide derivatives of xanthohumol and its metabolites products of hydroxylation, methylation, acetylation, epoxidation and cyclization of prenyl xanthohumol chain and its metabolites, sulphone xanthohumol derivatives, cyclization of chalcone to flavone, cyclization of chalcone to aurone and other products obtained as a result of chemical and microbiological attachment of sugars to xanthohumol and its metabolites is used.

In the use of the invention, the dose of prenylated chalcone derivative or its metabolites, preferably xanthohumol per one bee colony is from about 0.01 mg to about 1 g.

Prevention and/or controlling is carried out in bees and bumblebees. Preferably, bees of Apis mellifera and Apis ceranae species are selected.

In the use according to the invention, preferably a formulation mentioned above and defined in the claims is used.

The subject matter of the invention also relates to a formulation containing prenylated chalcone derivative or its metabolites for use in the method for preventing of collapse of insects from Apidae family.

The subject matter of the invention also relates to a formulation comprising prenylated chalcone derivative or its metabolites for the use in the method for preventing and/or treatment of diseases selected from Nosema spp. group, sacbrood disease, mycoses, Varroosis or other pathogens of insects from Apidae family.

The subject matter of the invention also relates to the formulation comprising prenylated chalcone derivative or its metabolites for use in the method for preventing or removing of effects of insecticides use from neonicotinoids group and other toxins in insects of Apidae family.

The term “about” used in the present specification both with regard to numerical values determining concentrations and doses typically and preferably should be understood as including values with a deviation of ±10% (a standard in biological studies) of indicated numerical value.

Insects of Apidae family include, among other ones, the insects of subfamily Apinae, tribe Apini, genus Apis, which in turn is divided into species known systematics as Apis ceranae, Apis dorsata, Apis florea and Apis mellifera. All species belonging to genus Apis live in organized societies. These insects feed on nectar, honeydew syrup and pollen. Their importance in biology and natural environment and in agricultural industry and fruit culture consists in pollination of flowers, what allows to obtain yield from fruit culture and other plant cultures.

Apidae family includes also insects looking like a large, stocky and thickly hairy bee which are named bumblebees. In systematics, they are described as bees of Bombus genus which includes also cuckoo bumblebees.

Morphological and molecular analyses showed that bees and bumblebees are very similar. Their biology and behaviour are similar in many aspects, but they differ for example by the fact that bees start flights at the temperature of 10° C., and bumblebees at 8° C. Compared to other insects, bumblebees are very efficient. Their significant merit is also the lack of communication system.

Research also showed that the structure of post-synaptic nicotine receptor of neural ends of the brain of those insects and other insects is very similar.

Chalcones (1,3-diaryl-2-prene) are derivatives of acetophenol and are intermediate compounds in biosynthesis of flavonoids. These are compounds of an open heterocyclic ring, and the cyclization of the ring gives a flavones configuration. The presence of chalcones has been found in liquorice (Glycorrhiza gabra), hop (Humulus lup ulus), helichrysums (Helichrysum arenarium) and willows (Salix sp.).

Prenylated chalcone derivatives are important group of metabolites, which may be extracted from hop cones and they include compounds such as xanthohumol (Xn), desmethylxanthohumol (DMX), isoxanthohumol (IX), 6-prenylonaryngenin (6-PN) and 8-prenylnaryngenine (6-PN) and 6-prenylonaryngenin (8-PN).

### Structure of Exemplary Chalcones

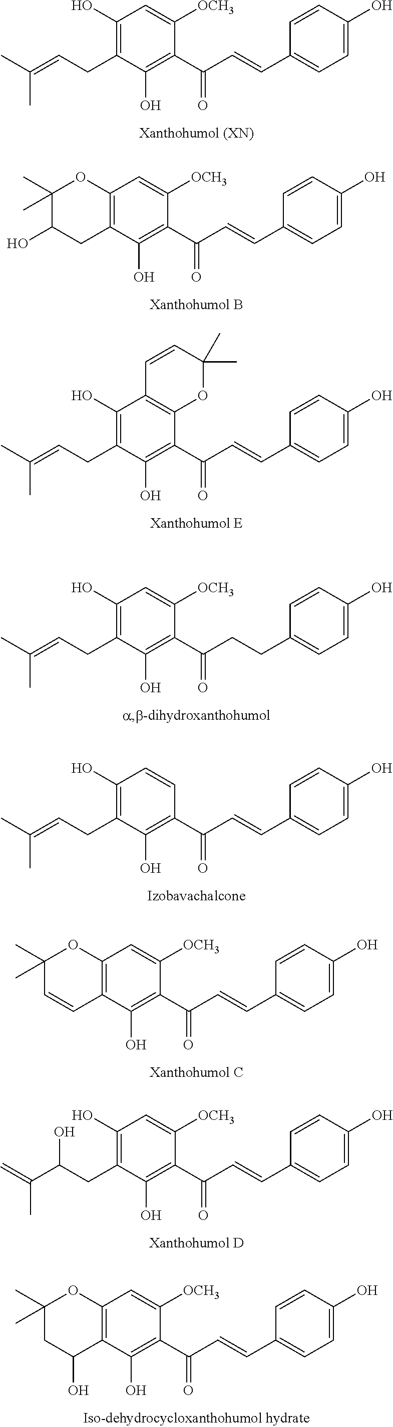

### Other Prenylated Chalcone Derivatives:

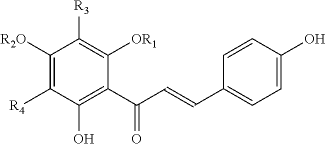

| | R1 | R2 | R3 | R4 |
|---|---|---|---|---|
| Desmetylxanthohumol | H | H | H | Prenyl |
| Xanthogalenol | H | Me | H | Prenyl |
| 4′-O-methylxanthohumol | Me | Me | H | Prenyl |
| 3′-geranyl | H | H | H | Geranyl |
| chalconaryngenin | | | | |
| 3′5′- | H | H | Prenyl | Prenyl |
| diprenylochalconaryngenin | | | | |
| 5′-prenyloxanthohumol | Me | H | Prenyl | Prenyl |
| Flavokavine | Me | Me | H | H |

### Xanthohumol Metabolites:

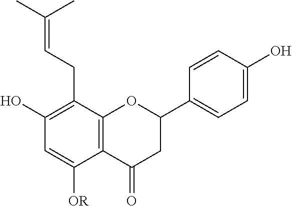

R=H, 8-prenylnaryngenin

### R=Me, Isoxanthohumol

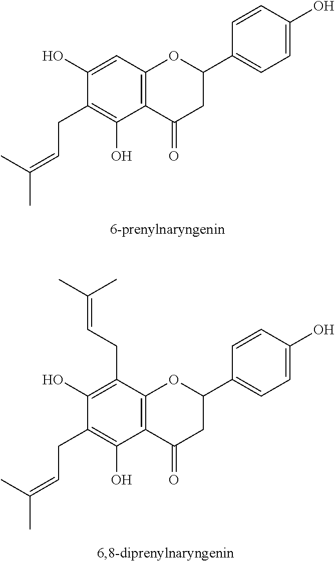 [SMILES: CC(C)=CCc1c(O)cc2c(c1O)C(=O)CC(c1ccc(O)cc1)O2 ; CC(C)=CCc1c(O)c(CC=C(C)C)c2c(c1O)C(=O)CC(c1ccc(O)cc1)O2]

Even though data concerning xanthohumol action on membrane receptors in insects have been very scarce so far, the studies carried out in animals show numerous uses of this compound.

In vitro and in vivo studies also show that xanthohumol reduces inflammatory and oxidative processes through various mechanisms. The most important are: removing free oxygen radicals and their reactive forms (RTF), inhibiting activity of cyclooxygensaes (COX I and COX II), a decreasing the production of prostaglandin, NO, TNFα, NFκB and counteracting peroxidation of membrane phospholipids.

Antioxidative and anti-inflammatory activity of xanthohumol is related to the presence of hydroxyl groups and the prenyl substituent in its molecule. α,β-unsaturated carbonyl moiety is also of high importance. It takes part in the Michel reaction with biologically important nucleophiles, e.g.: L-cysteine in proteins and with other molecules containing sulfhydryl groups. In this way, covalent adducts are formed which are the reason of loss of biological activity of different molecules bound by xanthohumol.

A wide spectrum of xanthohumol action results probably from the ability of this compound for binding biologically important molecules. The best known is the influence Xn on synthesis of polymerase a, topoisomerase I, alkaline phosphatase, aromatase, diacylglycerol acylotransferase, proteins of MAPK signalling pathway, FAK kinase, Akt and transcription factors STAT or NF-κB. A decrease of activity of these proteins induced by Xn causes pleiotropic cellular effects which are the consequence of proliferation disturbance, cellular cycle, adhesion and migration of neoplastic cells.

Noteworthing is also the information concerning its neuroprotective properties. In the model of focal ischaemia caused by MCAO in rats, which reflects focal brain ischaemia in vivo, xanthohumol significantly decreased the area of brain ischaemic necrosis. This suggests that xanthohumol may decrease neurotoxic effect induced also by β-amyloid peptide (Aβ). It is known that Aβ mediates in free radical mechanism and is one of the main reasons of Alzheimer's disease, increasing production of free radicals and lipid peroxidation in neural cells, which consequently leads to in their apoptosis.

Moreover, apart from the action on receptors, the mechanism of xanthohumol activity may be related to its selective activity upon lipid membranes. Studies on mechanisms of xanthohumol interaction with model lipid membranes indicate a potent modification of dynamic and structural properties of the membrane. Xanthohumol, even in very small concentrations, potently interacts with hydrophil fragments of the lipid membrane, influencing changes of bilayer thickness. It is also known that neonicotinoids, due to their high hydrophobicity, can very easily permeate lipid membranes. Xanthohumol interaction with lipid membrane may significantly limit its penetration by neonicotinoids, which, apart from the blockade of nicotine receptors may provide an explanation for its potent activity.

It was noticed during the realisation of studies presented in the present application that mechanism of protective activity of xanthohumol (Xn) is related to its neuroprotective action on nicotine′ receptors in insects consuming the formulation. Although this mechanism has not been fully investigated, it is known that neonicotinoids interact with nicotine receptors. However, after protection by xanthohumol, it was found that insects are elive, they become only partly paralysed but they do not lose their memory. It is possible that xanthohumol, apart the interaction with nicotine receptors and stiffening of neurone lipid membrane, has a protective influences on synaptic conduction, which finally causes the death of insects.

Xanthohumol (Xn), i.e. ((E-1-[2,4-dihydroxy-6-methoxy-3-(3-methylbut-2-enyl)-phenyl]-3-(4-hydroxyphenyl)-prop-2-en-1-on) is a natural compound from the prenylated chalcones group its main source is female inflorescence of Humulus Lupus. It is also found in beer in trace amounts. This compound is composed of two benzene rings (A and B) bound by α,β-unsaturated carbonyl system.

The presence of free hydroxyl group in its molecule results in easy isomerization to the typical flavone—an intermediate product in the biosynthesis pathway of flavonoids. Unlike other components contained in hop cones, xanthohumol is distinguished by higher lipophilicity, which is related to the presence of a prenyl subtituent in its structure. It is worth noting that in live biological systems, prenyl moieties (farnesyl or geranyl geranyl) allow numerous intracellular molecules to dock in the cell membrane and thus determine about their proper function. Similarly, it is suggested that in the case of xanthohumol, the moieties may significantly modulate biological activity of this compound, and they may also influence its physico-chemical properties and subcellular arrangement.

Xanthohumol exhibits poor water solubility. In order to dissolve it in an aqueous medium, different solvents known in the prior art may be used, including cyclodextrins. It is also possible to use ethanol solution of xanthohumol or xanthohumol dissolved in DMSO may be added to a syrup to obtain an active formulation.

The formulation according to the invention may be used as a protective preparation in the form of an additive to a syrup in the winter, spring or resting period (when waiting for the blooming of flowers of subsequent plants). The formulation may be added to a syrup, or produced as a ready-to-use preparation with the syrup and it may be use as a spray for bees, e.g. before placing hives in a pasture, that is suspected of pesticide contamination.

A very effective method of administration of the formulation to bees may also be dissolving it in the sugar syrup with appropriate sugar to water ratio and using it as a spray for Apidae, which consume the formulation when licking it from the body and wings. The preparation may also be poured into spaces between the frames in the hive.

The use of the formulation based on xanthohumol may contribute significantly to the protection of insects of Apidae family, and especially bees and bumblebees from an unfavourable influence of neonicotinoids and other toxins as well as infections with Nosemia spp and other microbiological threats. The problem is global, and that is why it deserves special attention. Until now a formulation of such a high effectiveness against bee collapse induced by environmental contamination in which the active substance is a natural compound has not been disclosed. Moreover, it is known on the basis of numerous literature data that xanthohumol is a compound that has a very beneficial influence on people's health.

Due to antimycotic and antibacterial activity of xanthohumol, the formulation also exhibits activity in the control of Nosema spp. (apis and cerenae). It was found on the basis of studies (initial results) that the amount of spores observed under a microscope after the use of Xn decreased by 60% on average in relation to control. Observation under a microscope of blended dead bees was related to counting of Nosema cells in the suspension of distilled water. The bees which did not consume the formulation with Xn contained 30±3 Nosema pieces, whereas after using the formulation for 1 week, their number decreased to 8±2 pieces of Nosema on average. The formulation was also investigated in several bee colonies in apiary infected with Nosemosis. It was found that after several days after the spraying with formulation containing xanthohumol, typical symptoms of Nosema spp. infection disappeared (soiling of frames and front wall of the hive with feces).

### BRIEF DESCRIPTION OF DRAWINGS

The invention will be further explained in detail on the basis of an Examples and Figures in which:

FIG. 1 shows a photo of a typically used small hive containing 100 bees.

FIG. 2 is a chart presenting the relationship between xanthohumol concentration in a syrup and viability of bees.

FIG. 3 is a comparative chromatogram of the purity of the obtained xanthohumol as compared to a standard. In the xanthohumol chromatogram (Xn), the control is a standard purchased from Sigma Aldrich. The lower panel shows chromatogram of xanthohumol obtained by own method from post-extraction waste of hop. The purity of the formulation used for preparing aqueous solution in syrup was over 99%.

FIG. 4 shows a typical bee frame used in a ˜40 000 hive with bees before imidaclopride spraying.

FIG. 5 A,B,C shows the inside of a control hive (without the protective action of xanthohumol formulation) 2 hours, 1 day and 13 days after the use of imidaclopride spraying.

FIG. 5A

The inside of the control hive 2 hours after the use of imidaclopride spraying. Almost all bees were paralyzed.

FIG. 5B

A frame from the control hive 1 day after the use imidaclopride spraying. Herding of bees together in swarms.

FIG. 5C

A frame from the control hive 13 days after the use of imidaclopride spraying. The bees still herd together in swarms.

FIG. 6 A, B, C show the inside of the hive where the bees took protective preparation with xanthohumol shown 2 hours after spraying, 1 day and 13 days after spraying.

FIG. 6A

The inside of the hive in which the bees took the protective formulation with xanthohumol, 2 hours after the use of imidaclopride spraying. Losses are seen, but compared to a the control hive, the picture is much different. The behaviour of bees indicated their better condition in relation to bees which did not take the protective formulation.

FIG. 6B

A frame from the hive in which bees were taking the protective formulation with xanthohumol (the picture was taken 1 day after the use of imidaclopride spaying). The bees did not leave brood and posted guards. In general the picture is similar to healthy bees.

FIG. 6C

A frame from the hive in which bees were taking the protective formulation with xanthohumol (the picture was taken days after the use of imidaclopride spraying). The bees were active as in normal hives. As may be seen (cells closed with wax and shining cells) they make honey in this case from goldenrod.

The invention is illustrated by the following examples:

### EXAMPLE 1

### A Study of the Influence of Different Xanthohumol Concentrations on the Viability of Bees with the Use of High Concentrations of Imidaclopride on Experimental Queen Cages

The experiment was carried out in Lublin, Poland in an apiary containing 250 bee colonies.

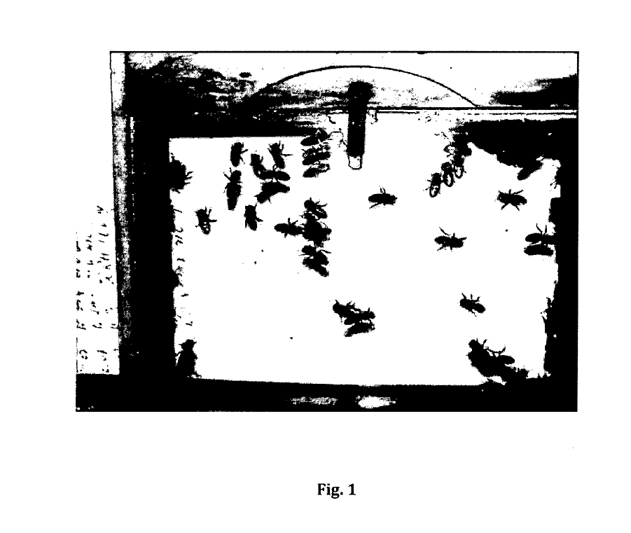

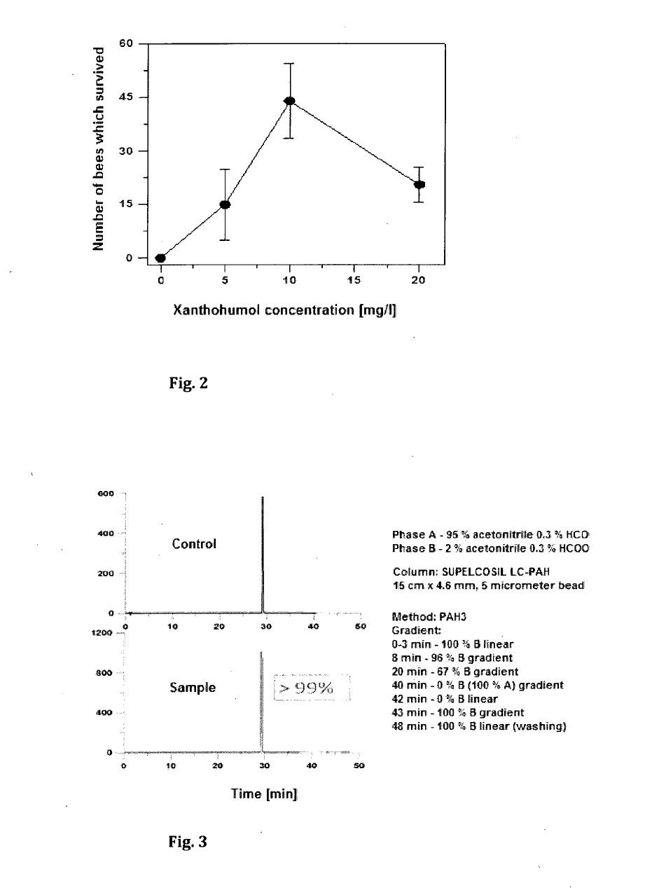

The influence of xanthohumol concentration in sugar syrup on the viability of bees was studied on samples of 100 bees placed in a special cage for studies, (FIG. 1). The bees were taking a protective formulation for three days in the form of xanthohumol dissolved in standard sugar syrup in three concentrations: 5, 10 and 20 mg of xanthohumol/1, respectively. Each concentration was studied in three independent small hives. After three days of taking the protective formulation, the bees received imidaclopride (in Kohinor 200 SL) in a concentration of 375 μl/l in a volume of ml of syrup (similar doses are used for controlling of insects in vegetable cultivations). As a control group, three small hives of 100 bees each were used, which were not fed with xanthohumol containing preparation. All xanthohumol concentrations and the control group were studied in thriplicate. Xanthohumol obtained by chromatographic method from hop post-extraction waste was used. The purity of the preparation was ˜99% (FIG. 3).

As shown in FIG. 2, there is a very clear relationship between the concentration of xanthohumol preparation in syrup and bee viability. At the concentration of 10 mg/l of xanthohumol, the viability of bees was highest and equal to ˜45%. Control bees, which were not fed with the formulation containing xanthohumol, died in 100%.

Xanthohumol purity plays an important role because a formulation of lower purity contains other hop compounds, e.g. humulons, the intensive smell of which may decrease attractivness of the preparation for bees. Moreover, in xanthohumol samples of lower purity, there are pesticides used in hop cultivation which may pass into honey.

### EXAMPLE 2

### A Study of the Influence of Xanthohumol in Syrup on the Viability of Bees with the Use of Low Concentration of Imidaclopride in Apiary

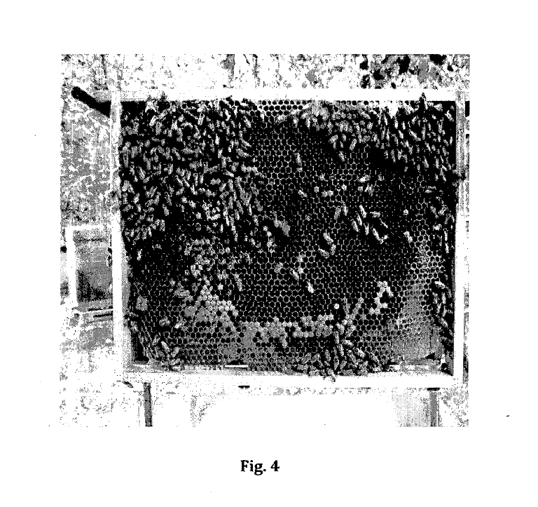

Similar studies as described in example 1 were carried out in three bee hives containing ˜40,000 bees. Frames with bees (FIG. 4) were sprayed with imidaclopride dissolved in water at a concentration of 125 μl/l (microlitres per litre). It is a half of imidaclopride concentration in Kohinor 200 SL used in cultivation of vegetables in soil. The purpose of the study was to investigate low doses of imidaclopride to which mainly are exposed the bees collecting nectar during agrotechnological procedures.

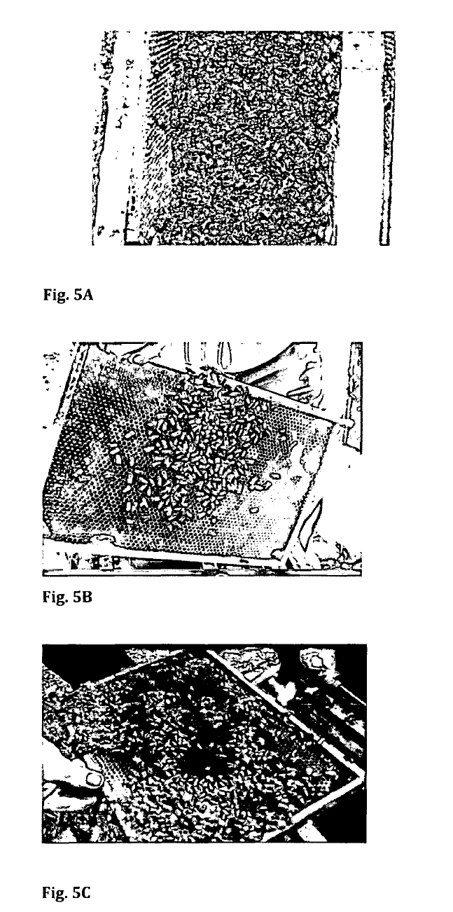

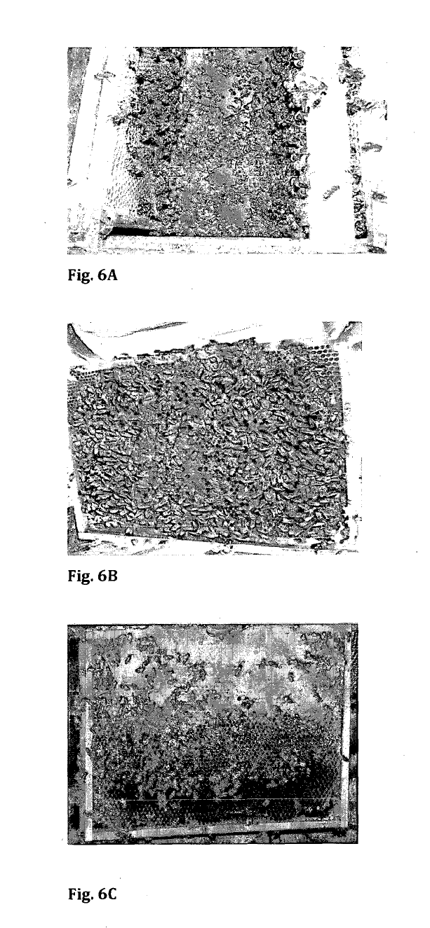

The bees were studied in two groups. The bees in control hives did not receive protection in the form of xanthohumol formulation before spraying with imidaclopride (FIGS. 5A, 5B and 5C). The studied group were bees which were given protective xanthohumol preparation in 10 mg/ml (milligram per litre) syrup for one week and then treated with imidaclopride spray at the above mentioned concentration (FIGS. 6A, 6B and 6C).

The results presented in FIGS. 5 and 6 show the behaviour of bees 2 hours, 1 day and 13 days after spraying in the control group (without protective preparation) and the studied group (with the protective preparation). Based on the above mentioned studies it may be concluded that in the case of bees which did not receive the protective xanthohumol formulation, imidaclopride significantly contributed to their death. It may be seen that almost all bees were paralyzed within two hours after spraying (FIG. 5A), 1 day after spraying, those bees which survived abandoned care over the brood and herded together in a large group called “a swarm” in apiary similar to that of winter time. Moreover, the bees were very weak and did not post guards in front of the hive (FIG. 5B). About a half of the population died. Even after two weeks from the procedure the bees still herded together, abandoning care over the brood. Their general condition indicated their great weakness which results in the lack of guards and bees remaining in the hive without flights to the pasture (for pollen). After 13 days from the administration of imidaclopride the bees still herded together in “swarms” (FIG. 5C), they did not fly outside the hive and did not produce honey. Their required stimulative feeding or otherwise they were dying.

The bees which took the formulation with xanthohumol behaved quite differently (FIG. 6A, B, C). As mentioned above, the syrup with xanthohumol was more attractive for them and during the first twenty four hours, they took 2.5 litre thereof. The use, like in the control group, of imidaclopride did not cause such drastic effects. Some bees died, ˜10%, but the remaining posted guards as soon as the next day and did not abandon care over the brood. The general appearance of the swarm indicated high efficacy of xanthohumol preparation. Partial collapse of the bees may be explained by the use of the preparation on frames, where imidaclopride acted also on the young bees which due to the hierarchy present in the hive may have not consumed sufficient amount of xanthohumol formulation. Three days from the spraying the bees started to fly to the posture (goldenrod flowers) and bring pollen which means that they did not lose memory. Two weeks after the administration of imidaclopride, the bees from both hives treated with protective formulation did not show any differences in behaviour compared to the healthy hives.

### EXAMPLE 3

A study of the influence of different concentrations of xanthohumol on the viability of bees with the use of poisonous biocide thiamethoxam (thiamethoxam 25%) (Aktara)

The experiment was carried out in Loma-Zawady Przedmieście, Poland in an apiary consisting of 25 colonies based in hives of Dadant type (frame 435×300 mm). The hives had wire-net, detachable bottom in the form of a diagnostic tray. The nest body of the hives consisted of 10 frames.

Bee colonies in the form of separated colonies were purchased from local producer. Each separated colony consisted of 3 frames of brood and frames with supply of bee bread and honey. All frames were settled in “black” by bees.

The experiments were carried out on 25 swarms of bees divided into VI groups. Groups I to V, consisted of 4 hives, each.

Group I-V

In groups I-V, xanthohumol at the concentration of 40 mg/l was used as a protective formulation. Actara at the concentration of 0.4 g/l was used for poisoning of insects (the concentration used in spraying of beetle).

Group I—the control sample included bee colonies not subjected to any procedures.

Group II—bee colonies poisoned with Actara, but not protected with the formulation containing xanthohumol.

Group III—bee colonies treated with the protective formulation in the form of spray without poisoning with Actara. 20 mg of xanthohumol were dissolved in 1 ml of ethyl alcohol and subsequently added to 0.5 l of sugar syrup in water in a ratio of 1:1, and four hives were sprayed with use of a spraying machine so that one bee colony received a dose of about 5 mg of xanthohumol. Actara was not used in this experiment.

Group IV—bee colonies treated with the protective preparation in the form of spray. Poisoning with Actara by alimentary route. 20 mg of xanthohumol were dissolved in 1 ml of ethyl alcohol and subsequently added to 0.5 l of sugar syrup in water in a ration 1:1 and four hives were sprayed with spraying machine so that one bee colony received a dose of 5 mg of xanthohumol. Poisoning with Actara given with 1:1 syrup (1 kg of sugar+1 l of water).

Group V—bee colonies treated with the protective preparation in the form of spray. Poisoning with Actara by contact route. 20 mg of xanthohumol were dissolved in 1 ml of ethyl alcohol and subsequently added to 0.5 l of sugar syrup in water in a ratio 1:1 and four hives were sprayed with the spraying machine so that one bee colony received a dose of about 5 mg of xanthohumol. Poisoning was carried out in the form of small-molecule spraying which was used for each frame with bees.

Group VI (a-d)

In this group, a formulation of different xanthohumol concentrations was used, and Actara (thiametoxam 25%) for poisoning of bees, at a dose of 0.2 g/l (concentration used by fruit farmers) was used. The control group were bees subjected only to spraying procedure with Actara.

Group VI a)—bees were protected with the formulation of xanthohumol concentration of 0.08 mg/l.

0.01 mg of xanthohumol was dissolved in 1 ml of ethyl alcohol and subsequently added to 0.125 l of sugar syrup in water in a ratio 1:1 and 1 hive was sprayed so that one a bee colony received a dose of about 0.01 mg of xanthohumol. After 24 hours, the bee colony was poisoned by a contact route. The poisoning was carried out in the form of small-molecule spraying which was used for each frame with bees.

Group VI b)—the bees were protected with the preparation of xanthohumol in concentration 1.6 mg/l.

0.2 mg of xanthohumol was dissolved in 1 ml of ethyl alcohol and subsequently added to 0.125 l of sugar syrup in water in a ratio 1:1 and 1 hive was sprayed so that one bee colony received a dose of about 0.2 mg of xanthohumol. After 24 hours, the bee colony was poisoned by the contact route. Poisoning was carried out in the form of small-molecule spraying which was applied on each frame with the bees.

Group VI c)—the bees were protected with a formulation of xanthohumol in concentration 16 mg/l.

2 mg of xanthohumol dissolved in 1 ml of ethyl alcohol which was added to 0.125 l of sugar syrup in water in a ration of 1:1 in duplicate and it was sprayed on 2 hives so that one bee colony received a dose of about 2 mg of xanthohumol. After 24 hours, the bee colonies were poisoned by the contact route. The poisoning was carried out if the form of a small-molecule spray which was applied on each frame with bees.

Group VI d)—the bees were protected with a preparation of xanthohumol concentration 8 g/1.

1 g of xanthohumol dissolved in 10 ml of ethyl alcohol which was then added to 0.125 l of sugar syrup in water in a ratio 1:1 and it was sprayed on 1 hive so that one bee colony received a dose of about 1000 mg of xanthohumol (1 hive). After 24 hours bee colony was poisoned by a contact route. Poisoning was carried out in the form of small-molecule spray which was applied on each frame with bees.

Observations and Conclusions from Studies in Groups I-VId

In group I, no abnormalities were observed in the behavior of bees—standard healthy bee colonies.

In group II, where protective preparation in the form of xanthohumol was not used and the bees were poisoned with Actara at a concentration of 0.4 g/l, all colonies died after 15 minutes showing symptoms of poisoning.

In group III, where Actara was not used, but the bees were sprayed only with the protective formulation at a concentration of xanthohumol 40 g/l, it was observed that the formulation containing xanthohumol improved the behaviour of bee colony and significantly influenced hygienic instinct of a bee. After protecting the colonies with the formulation, a significantly higher amount of contamination was observed on the diagnostic tray compared to the control colonies. Dead mites Varroa destructor were also observed among contaminations, which was not observed in the control hives. As a result of increasing the hygienic instinct induced by the formulation the formulation stimulated the bees to bite Varroa destructor mites.

In group IV, where bees were poisoned with Actara at a concentration of 0.4 g/l (alimentary route) after 24 hours from the use of the protective preparation at the concentration of xanthohumol of 40 mg/l losses were observed at the level of 35% (measurements were conducted by weighing on a balance dead bees dropped on the bottom and in front of the hive)). Colonies poisoned by alimentary route, which were alive after 24 hours started to fly for pollen again. They did not lose memory (a failure to carry pollen is related to memory loss). All bees protected with the preparation containing xanthohumol, and then poisoned, survived the withdrawal period of the agent. On the day on which the withdrawal period expired, all poisoned and previously protected colonies carried pollen and showed increased movement at the beehive entrance.

In group V, where poisoning by the contact route was applied after 24 hours from the use of the protective formulation of xanthohumol in concentrations 40 mg/l the losses at the level of 10% were observed. Colonies poisoned by a contact route which survived after 24 hours, re-started flights for pollen. They did not lose memory and carried pollen. All colonies protected with the preparation containing xanthohumol, and then poisoned, survived the withdrawal period of the agent. On a day when the withdrawal period expired, all poisoned and previously protected colonies carried pollen and showed an increased movement at the outlet of the hive.

In the control group for groups VIa-VId where the protective preparation was not used and the bees were poisoned with Actara at a concentration of 0.2 g/l, all bees collapsed after several dozen minutes showing symptoms of poisoning.

Where xanthohumol at a concentration of 0.08 mg/l was used as a protective preparation (0.01 mg per colony) (Group VI a) and the poisoning with Actara 0.2 g/l was carried out after 24 hours from protection, it was observed that 60% of bee colony did not survive the procedure. After the use of the protective preparation in the amount of 0.01 mg per colony, no symptoms increasing the vigour as the described in group III were observed. The bees which survived did not restart flights for pollen either after 24 hours or 48 hours, however, herding of the bees together in swarms was observed. The behaviour of the surviving bees resembled the one presented in Example II, (the poisoned control bees herded together in swarms and did not leave the hive). In order to strengthen the vigour of the remaining 40% population of bee colony, spraying with the preparation containing xanthohumol was carried out at a concentration of 1.6 mg/l where the discussed bee colony received the dose of 2 mg. After 48 hours, the bees restarted flights around the hive. After a week from spraying with the preparation containing xanthohumol, the behaviour of the colony returned to normal, which means that the bees carried pollen. After three weeks from poisoning, the bees did not differ from the control. It means that the formulation containing xanthohumol may be also used in cases of already existing poisoning.

Where a concentration of 1.6 mg/l of xanthohumol (0.2 mf per colony) was used (Group VI b), about 40% of the bee colony did not survive the procedure. However, after 48 hours, the bees that survived, restarted flights for pollen.

Where a concentration of 16 mg/l of xanthohumol (2 mg per colony) was used (Group VI c), almost all bees survived the procedure and there were few losses. The bees that survived after 24 hours, restarted flights for pollen. After seven days from poisoning it was found that the protected bees which survived do not show any symptoms of poisoning.

Where a concentration of 8 g/l of xanthohumol (1 g per colony) was used (Group VI d), 90% of the bees poisoned with the pesticide and protected with the formulation 24 hours earlier survived; only 10% losses were observed. When the hive was inspected 24 hours after the xanthohumol preparation use before poisoning with the pesticide, it was found that the dose of 1 gram per a hive (the preparation of a concentration of 8 g/l), which exceeded 500 times the beneficial dose of 2 mg (2 g/l), did not cause any toxic effects. No dead bees were found on the bottom. After seven days, the bees did not show any symptoms of poisoning.

After using the preparation according to the invention in all studied concentrations of xanthohumol, an increased movement of bees was observed at the beehive entrance and a defense of the hive was increased, and, moreover, the increased noise in the hive was observed as well as increased flights of the bees compared to flights from the control hives. In the first half of August, in the hives protected with the preparation containing xanthohumol, a significant increase in of brooding of bee mothers was observed compared with the control. This indicates a significant increase of bee colony power before winter, which significantly improves its survival.

Xanthohumol formulation is attractive for the bees with regard to its smell and taste. Taste: the bees quickly and readily take it in a sugar solution. Smell: an effective attempt to rob the colony where the formulation in syrup was administered, despite protection.

Other Studies Carried Out with the Use of the Formulation Containing Xanthohumol Conducted in Poland

The studies prowed that the formulation containing xanthohumol (at a dose of 5 mg per one bee colony) may be used also for the treatment of the virus of sacbrood disease and mycosis and Nosemosis. In the case of sacbrood disease after protection, the colony started to work. However, in the case of mycosis and Nosemosis, fast purification of the nest from dead insects occurred. The increased hygienic instinct was shown by studies carried out on bee colonies by a dozen of beekeepers in Poland.

After the use of the preparation containing xanthohumol in colonies with viral sacbrood disease, a quiet exchange of the mother occurred. The preparation may be an agent of diagnostic value. Thus, xanthohumol may trigger mechanisms leading to a diagnosis of the mother's disease (as a vector of transfer) with concomitant signal for its quiet exchange.

Furthermore, it was found on the basis of the studies with the use of the formulation according to the invention, that there is a possibility of rehabilitating the protected bees in these regions which were contaminated by pesticides, where it has not been possible so far. In the hive where a control bee colony unprotected with the preparation was stocked with new unprotected colony, it collapsed within a short time. A hive contaminated with a pesticide is poisonous for bees even after several weeks. However, after putting into the hive a colony protected with the formulation of xanthohumol dissolved in syrup in concentration 2 g/l by spray, the colony still lived and did not show symptoms of poisoning.

1. A formulation preventing collapse of insects of Apidae family and increasing their vigour characterised in that it comprises a prenylated chalcone derivative or its metabolites of purity about 0.001-100%, a solvent and additional substances acceptable in apiary in a concentration of prenylated chalcone derivative or its metabolite not less than 0.001 mg/l of the formulation.

2. A formulation preventing collapse of insects of Apidae family and increasing their vigour characterized in that it comprises a prenylated chalcone derivative or its metabolites of purity about 0.001-100%, a solvent and additional substances acceptable in apiary in a concentration of prenylated chalcone derivative or its metabolite 0.001-1000 mg/1 l of the formulation.

3. The formulation according to claim 1 or 2, which comprises as a prenylated derivative a compound selected from the group consisting of xanthohumol, xanthohumol B, xanthohumol E, α,β-dihydroxanthohumol, isobavachalcone, xanthohumol C, xanthohumol D, isodehydrocycloxanthohumol hydrate, desmethylxanthohumol, xanthogalenol, 4′-O-methylxanthohumol, 3′-geranylchalconaryngenin, 3′,5′-diprenylchalconaryngenin, 5′-prenylxanthohumol, flavocavine and/or other prenylated chalcone derivatives.

4. The formulation according to claim 1 or 2 which contains xanthohumol as a prenylated derivative of chalcone.

5. The formulation according to any of claim 1 or 3-4 wherein the concentration of prenylated chalcone derivative or its metabolite, preferably xanthohumol, in the formulation is not less than 0.001 mg/l.

6. The formulation according to claim 1 wherein the concentration of prenylated chalcone derivative or its metabolite, preferably xanthohumol, in the formulation is in the range of about 0.08 mg/l to about 8 g/l.

7. The formulation according to claim 6 in wherein xanthohumol concentration in the formulation is selected from a group including about 0.08 mg/l, 0.1 mg/l, 0.5 mg/l, 1 mg/l, 1.6 mg/l, 2 mg/l, 5 mg/l, 10 mg/l, 16 mg/l, 20 mg/l, 30 mg/l, 40/ml, 50 mg/l, 70 mg/l, 100 mg/l, 200 mg/l, 300 mg/l, 1 g/l, 5 g/l or 8 g/l.

8. The formulation according to claim 2 wherein the concentration of prenylated chalcone derivative or its metabolite, preferably xanthohumol, in the formulation is in the range of about 0.08 mg/l to about 1 g/l, preferably 1-40 mg/l.

9. The formulation according to any of claims 1-8 which contains xanthohumol of purity from 0.001 to 100%.

10. The formulation according to claim 8 which contains xanthohumol of purity 0.1%, 1%, 5%, 10%, 20%, 26%, 30%, 40%, 50%, 60%, 70%, 80% or preferably at least 90%.

11. The formulation according to claim 1 or 2, which as a metabolite of prenylated chalcone derivative, comprises a compound selected from a group consisting of isoxanthohumol, desmethylxanthohumol, 6-prenylaryngenin, 8-prenylaryngenin, 6,8-prenylareyngenin, glucuronide derivatives of xanthohumol and its metabolites, hydroxylation, methylation, acetylation, epoxidation and cyclization of prenylated chain of xanthohumol and its metabolites products, sulfone xanthohumol derivatives, cyclization of chalcone to flavanone, cyclization of chalcone to aurone and other products derived as a result of chemical and microbiological attachment of sugars to xanthohumol and its metabolites.

12. The formulation according to claim 1 or 2 which contains sugar syrup as an additional substance.

13. The formulation according to any of claim 1-12 which, comprises as an additional substance, the substance selected from the group consisting of other flavonoids and chalcones or their metabolites, coumarins, carotenoids, polyphenols, oxalic acid, formic acid, ascorbic acid, fatty acids, lipids, liposomal forms of the above mentioned compounds, proteins, peptides, vitamins, minerals, propolis, honeys, flower pollens, royal jelly, plant oil, derivatives of castor oil, decosahexanoic acid, anise oil or eucalyptus oil, tea extracts, hop extracts, coffee extracts, caffeine or other herbal or fruit and vegetable extracts.

14. The formulation according to claim 1 or 2 which contains aqueous solution as a solvent.

15. The formulation according to claim 1 or 2 which contains pure alcohol or aqueous solution of ethyl alcohol as a solvent.

16. The formulation according to claim 1 or 2 which contains aqueous solution of DMSO, Kolliphor or Cremophor as a solvent.

17. The formulation according to claim 1 or 2 which contains cyclodextrins and/or sugars or their complexes as a solvent.

18. The formulation according to claim 1 or 2 wherein non-polar solvent is used as a solvent.

19. The formulation according to claim 14 wherein aqueous solution is an alkaline solvent.

20. The formulation according to claim 14 wherein aqueous solution is an acidic solvent.

21. The formulation according to claim 14 wherein aqueous solution is a solvent containing complexes of prenylated chalcone derivative or its metabolite, preferably xanthohumol, with alkaline and/or transitory earth metals.

22. The formulation according to any of claims 1-21 in solid or liquid form.

23. The formulation according to claim 22 in the form of sugar syrup, dough to feed insects, in the form of spray or for applying to spaces between the frames in the hive.

24. The formulation according to claim 22, in the form of sugar strip or other strip of nutritional substances for biting by insects.

25. The formulation according to claim 22 in the form of a strip on a matrix made of paper or plastics.

26. The formulation according to any of claims 1-25 for use at a dose of about 0.01 mg to about 1 g for one bee colony.

27. The use of prenylated chalcone derivative or its metabolites in the prevention and/or control collapse of insect from Apidae family as a result of collapse disease (CCD) of bees and bumblebees.

28. The use according to claim 27 where as prenylated chalcone derivative, a compound is used selected from a group consisting of xanthohumol, xanthohumol B, xanthohumol E, α,β-dihydroxanthohumol, izobavachalcone, xanthohumol C, xanthohumol D, isodehydrocyclo xanthohumol hydrate, desmethylxanthohumol, xanthogalenol, 4′-O-methylxanthohumol, 3′-geranyl chalconaryngenin, 3′,5′-diprenylchalconaryngenin, 5′-prenylxanthohumol, flavokavine and/or other prenylated chalcone derivatives.

29. The use according to claim 28 where, xanthohumol is used as prenylated chalcone derivative.

30. The use according to claim 27 where as metabolite of prenylated chalcone derivative, a compound selected from a group consisting of isoxanthohumol, desmethylxanthohumol, 6-prenylnaringenin, 8-prenylnaringenin, 6,8-prenylnaringenin, glucuronide derivatives of xanthohumol and its metabolites, products of hydroxylation, methylation, acetylation, epoxydation and cyclization of prenylated xanthohumol chain and its metabolites, sulfone xanthohumol derivatives, cyclization of chalcone to flavons, cyclization of chalcone to aurone and other products obtained as a result of chemical and microbiological attachment of sugars to xanthohumol and its metabolites is used.

31. The use according to any of claims 27-30, wherein the dose of prenylated chalcone derivative or its metabolite, preferably xanthohumol per one bee colony is from about 0.01 mg to about 1 g.

32. The use according any of claims 27-31 wherein prenylated chalcone derivative or its metabolite is used in bees and bumblebees.

33. The use according to claim 32 wherein bees are selected from Apis mellifera and Apis cerenae species.

34. The use according to any of claims 27-33, wherein the formulation is used as defined in any of the claims 1-26.

35. The use of prenylated chalcone derivative or its metabolites for improving the vigor of insects from Apidae family.

36. The use according to claim 35 wherein as prenylated chalcone derivative, a compound from a group consisting of xanthohumol, xanthohumol B, xanthohumol E, α,β-dihydroxanthohumol, isobavachalcone, xanthohumol C, xanthohumol D, isodehydrocycloxanthohumol hydrate, desmethylxanthohumol, xanthogalenol, 4′-O-methylxanthohumol, 3′-geranyl chalconaryngenin, 3,5′-diprenylchalconaryngenin, 5′-prenylxanthohumol, flavokavine and/or other prenylated chalcone derivative is used.

37. The use according to claim 36 wherein as a prenylated chalcone derivative, xanthohumol is used.

38. The use according to claim 35 wherein as a metabolite of prenylated chalcone derivative, a compound selected from a group consisting of isoxanthohumol, desmethylxanthohumol, 6-prenylnaryngenin, 8-prenylnaryngenin, 6,8-prenylnaryngenin, glucuronide derivatives of xanthohumol and its metabolites, products of hydroxylation, methylation, acetylation, epoxydation and cyclization of prenyl xanthohumol chain and its metabolites, sulfone xanthohumol derivatives, cyclization of chalcone to flavone, cyclization of chalcone to aurone and other products obtained as a result of chemical and microbiological attachment of sugars to xanthohumol and its metabolites is used.

39. The use according to any of claims 35-38 wherein xanthohumol dose per one bee colony is from about 0.01 mg to about 1 g.

40. The use according to any of claims 35-39 wherein prenylated chalcone derivative or its metabolite is used in bees and bumblebees.

41. The use according to claim 40 wherein bees are selected from Apis mellifera and Apis cerenae species.

42. The use according to any of claims 35-41 wherein the formulation is used as defined in claims 1-26.

43. The use of prenylated chalcone derivative or its metabolites for control of diseases selected from a group of Nosema spp., sac..disease, mycoses, Varroosis or other pathogens of insects of Apidae family.

44. The use according to claim 43 wherein as prenylated chalcone derivative, a compound selected from a group consisting of xanthohumol, xanthohumol B, xanthohumol E, α,β-dihydroxanthohumol, isobavachalcone, xanthohumol C, xanthohumol D, isodehydrocycloxanthohumol hydrate, desmethylxanthohumol, xanthogalenol, 4′-O-methylxanthohumol, 3′-geranyl chalconaryngenin, 3,5′-diprenylchalconaryngenin, 5′-prenylxanthohumol, flavokavine and/or other prenylated chalcone derivative is used.

45. The use according to claim 44 wherein xanthohumol is used as prenylated chalcone derivative.

46. The use according to claim 43 wherein as a metabolite of prenylated chalcone derivative, a compound selected from a group consisting of isoxanthohumol, desmethylxanthohumol, 6-prenylnaryngenine, 8-prenylnaryngenine, 6,8-prenylnaryngenine, glucuronide derivatives of xanthohumol and its metabolites, products of hydroxylation, methylation, acetylation, epoxydation and cyclization of prenylated xanthohumol chain and its metabolites, sulfone xanthohumol derivatives, cyclization of chalcone to flavone, cyclization of chalcone to aurone and other products obtained as a results of chemical and microbiological attachment of sugars to xanthohumol and its metabolites is used.

47. The use according to any of claims 43-46 wherein the dose of prenylated chalcone derivative or its metabolite, preferably xanthohumol per one bee colony is from about 0.01 mg to about 1 g.

48. The use according to any of claims 43-47 wherein prenylated chalcone derivative is used in bees.

49. The use according to claim 48 wherein the bees are selected from Apis mellifera and Apis cerenae species.

50. The use according to any of claims 43-49 wherein the formulation as defined in any of claims 1-26 is used.

51. The use of prenylated chalcone derivative or its metabolites for the prevention and/or control of the effects of the use of insecticides from neonicotinoids group and other toxins in insects of Apidae family.

52. The use according to claim 51, wherein as prenylated chalcone derivative, a compound of the group consisting of xanthohumol, xanthohumol B, xanthohumol E, α,β-dihydroxanthohumol, isobavachalcone, xanthohumol C, xanthohumol D, isodehydrocycloxanthohumol hydrate, desmethylxanthohumol, xanthogalenol, 4′-O-methylxanthohumol, 3′-geranyl chalconaryngenin, 3,5′-diprenylchalconaryngenine, 5′-prenylxanthohumol, flavokavine and/or other prenylated chalcone derivative is used.

53. The use according to claim 52 wherein xanthohumol is used as prenylated chalcone derivative.

54. The use according to claim 51 wherein as metabolite of prenylated chalcone derivative, a compound selected from a group consisting of isoxanthohumol, desmethylxanthohumol, 6-prenylnaryngenin, 8-prenylnaryngenin, 6,8-prenylnaryngenin, glucuronide derivatives of xanthohumol and its metabolites, products of hydroxylation, methylation, acetylation, epoxydation and cyclization of prenylated xanthohumol chain and its metabolites, sulfone xanthohumol derivatives, cyclization of chalcone to flavones, cyclization of chalcone to aurone and other products obtained as a result of chemical and microbiological attachment of sugars to xanthohumol and its metabolites is used.

55. The use according to any of claims 51-54 wherein the dose of prenylated chalcone derivative or its metabolite, preferably xanthohumol per one bee colony is from about 0.01 mg to about 1 g.

56. The use according to any of claims 51-55 wherein prevention and/or control is carried out in bees and bumblebees.

57. The use according to claim 56 wherein the bees are selected from Apis mellifera and Apis cerenae species.

58. The use according to any of claims 51-57 wherein the formulation as defined in any of claims 1-26 is used.

59. The formulation containing prenylated chalcone derivative or its metabolites for use in a method of preventing of collapse of bees of Apidae family.

60. The formulation comprising prenylated chalcone derivative or its metabolites for use in a method of prevention and/or treatment of diseases selected from a group Nosema spp., sacbrood disease, mycoses, Varroosis or other pathogens of insects of Apidae family.

61. The formulation containing prenylated chalcone derivative or its metabolites for use in a method of preventing or removing of the effects of the use of insecticides of neonicotinoids group and other toxins in insects of Apidae family.
